(19) United States
(12) Patent Application Publication
(10) Pub. No.: US 2012/0059422 A1
(43) Pub. Date: Mar. 8, 2012

(54) METHODS FOR COMPRESSION FRACTURE TREATMENT WITH SPINOUS PROCESS FIXATION SYSTEMS

(75) Inventor: Phillip G. Esce, Spartanburg, SC (US)

(73) Assignee: LANX, INC., Broomfield, CO (US)

(21) Appl. No.: 13/214,875

(22) Filed: Aug. 22, 2011

Related U.S. Application Data
(60) Provisional application No. 61/379,632, filed on Sep. 2, 2010.

(51) Int. Cl. A61B 17/88

(52) U.S. Cl. 606/279

(57) ABSTRACT

Embodiments presently disclosed generally relate to the treatment of spinal fractures, such as spinal compression fractures. In one embodiment, a method of treating a spinal fracture includes identifying a vertebra having an untreated fracture, coupling a first device to the spinous processes of the vertebra with the fracture and a vertebra positioned directly superior to the fractured vertebra, and coupling a second device to the spinous processes of the vertebra with the fracture and the vertebra directly inferior to the fractured vertebra.

### BACKGROUND

The human spine contains a series of bony segments separated by discs and coupled together with muscle, ligaments, and other connective tissues. A large number of ailments may afflict one or more of these components. One such ailment involves a fracture of the vertebral body, and in particular, a compression fracture. Such a fracture may result in the compression of the bone into itself, resulting in pain and misaligned vertebral segments.

The treatment of vertebral compression fractures, to date, often involves injecting bone cement or some other filler into the vertebral body. The goal of such treatment is to reshape the vertebral body into something more similar to its non-compressed state. While successful for some patients, unfortunately this approach does not help many patients who continue to suffer from pain and other symptoms.

### BRIEF SUMMARY

Embodiments presently disclosed generally relate to the treatment of spinal fractures, such as spinal compression fractures. In one embodiment, a method of treating a spinal fracture includes identifying a vertebra having an untreated fracture, coupling a first device to the spinous processes of the vertebra with the fracture and a vertebra positioned directly superior to the fractured vertebra, and coupling a second device to the spinous processes of the vertebra with the fracture and the vertebra directly inferior to the fractured vertebra.

The untreated fracture may include a compression fracture, which has reduced the height of the vertebra by a certain amount, such as at least about fifty percent (50%), at least about seventy-five percent (75%), at least about ninety percent (90%) and the like. If desired, a bone cement may be applied to the untreated fracture of the vertebra after coupling the first device to the spinous processes.

The devices may include a wide range of structures capable of being coupled to and/or between adjacent spinous processes. In one embodiment, the device includes a first member having first and second wings, with the first wing for coupling to the spinous process and the second wing for coupling to the superior vertebra. A second member, having wings may similarly be coupled to the spinous process and the superior vertebra, with an extension member adapted to extend between the spinous processes. In some embodiments, bone growth promoting substances are placed between the spinous processes, and may be inserted in the extension member between the spinous processes. The device may have first and second members adapted to couple to opposing lateral sides of the spinous process, and to opposing lateral sides of an adjacent spinous process. In a particular embodiment, the method includes maintaining a distance between the spinous processes prior to coupling the first device thereto.

The disclosure further provides methods of treating a previously untreated spinal compression fracture. In one embodiment a vertebra is identified having an untreated compression fracture and a spinous process. A first device is selected for coupling to the spinous process and to a spinous process of a second vertebra adjacent the vertebra. The first device includes a first component having a central portion and two extension portions extending from the central portion in generally opposite directions, with the two extension portions being generally coplanar and having separate longitudinal axes. The first device further includes a second component having a plurality of spikes, an intermediate member adapted to be disposed between the spinous process and the spinous process of the second vertebra, and a locking member adapted to couple the first and second components together with the intermediate member, wherein the first and second components are positioned on opposing lateral sides of the spinous process when coupled together. The method includes coupling the first device to the spinous processes to hold the vertebra in a set position relative to the second vertebra. A similar device may be selected and coupled to the spinous process and to a spinous process of a third vertebra adjacent the vertebra, whereby the fractured vertebra is positioned between the second and third vertebrae.

In some embodiments, the methods include maintaining a distance between the spinous process and the spinous process of the second vertebra after the coupling of the first device to the spinous process and the spinous process of the second vertebra. The coupling of the first and/or second devices to two adjacent spinous processes may be accomplished without distracting a gap between the spinous processes.

This summary provides only a general outline of some embodiments disclosed herein. Many other objects, features, advantages and other possible modifications to the disclosed embodiments will become more fully apparent from the following detailed description, the appended claims and the accompanying drawings.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a side view of three adjacent vertebra, with the middle vertebra having a compression fracture;

FIG. 2 is a side view of the vertebra shown in FIG. 1, with a device coupled to the upper and middle vertebra;

FIG. 3 is a side view of the vertebra shown in FIG. 1, with devices coupling the three vertebra;

FIG. 4 is a posterior cross sectional view of an implant according to an embodiment;

FIG. 5 is a side elevational view of the implant of FIG. 4 in situ;

FIG. 6 is a an exploded perspective view of the implant of FIG. 4;

FIG. 7 is a posterior elevational view of the implant of FIG. 4;

FIG. 8 is an anterior elevational view of the implant of FIG. 4;

FIG. 9 is a top plan view of the implant of FIG. 4;

FIG. 10 is a posterior elevational view of the implant of FIG. 4 showing the assembly in an alternate position;

FIG. 11 is a side elevational view of the implant of FIG. 4; and

FIG. 12 is a perspective view of a pair of implants like that of FIG. 4 in situ.

### DETAILED DESCRIPTION

Embodiments presently disclosed generally relate to methods and systems for treating spinal compression fractures, and in particular, to methods and systems for treating previously untreated vertebral compression fractures.

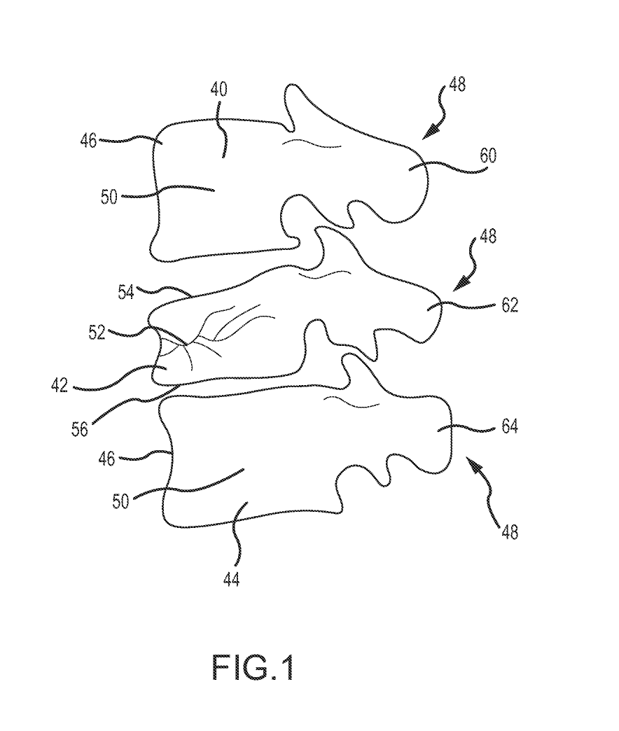

FIG. 1 illustrates three vertebrae 40, 42, 44 each with an anterior side 46, a posterior side 48 and lateral sides 50 (only one shown). For convenience of illustration, the discs between vertebrae 40, 42 and 44 are not included in the Figures. Vertebrae 40 and 44 are fully intact, while vertebra 42 has a vertebral compression fracture (VCF) 52. The VCF 52 has resulted in a superior surface 54 and an inferior surface 56 of vertebra 42 being displaced towards each other. The force required to reduce the VCF 52 (i.e., to displace superior surface 54 and inferior surface 56 of the vertebra 42 back to their original positions) can often be rather high. Present needles for use within vertebrae can bend or deform in the presence of lateral force, and thus, often are not rigid enough to reduce VCF's. Balloons can be placed in the fractured vertebra and expanded to reduce the VCF. Such balloons, however, will expand equally in all radial directions, which can cause the vertebra to shatter on the anterior, posterior, or lateral sides.

Other techniques for attempting to reduce the VCF suffer from similar problems, and are unsuccessful in treating a large percentage of patients. For example, the procedures known as “kyphoplasty” and vertebroplasty can have a success rate of less than seventy percent (70%).

In lieu of attempting to restore vertebra 42 to its normal height through common techniques, providing a posterior tension band or structure without the need for distraction of the vertebra can produce superior results. This is accomplished, in some embodiments, by coupling spinal implants to the spinous processes of three adjacent vertebrae, with the compressed or fractured vertebra in the middle of the three. For example, if T12 is the compressed vertebra, a spinal implant such as those described herein, is coupled between T11-T12 and a second spinal implant is coupled between T12-L1. The spinal implants may be, for example, eight millimeters (8 mm), or ten millimeters (10 mm), or twelve millimeters (12 mm) in height. Such sizes typically can provide the proper posterior tension band characteristics and do not distract the vertebra to which they are coupled. In a preferred embodiment, the spinous processes of the treated spinal segments are not spread apart, but instead the implants provide posterior support.

Embodiments of the present disclosure can be used to treat VCFs for which a repair has already been attempted (i.e., a prior surgical attempt). Embodiments of the present disclosure are particularly useful in treating VCFs which have not undergone a prior surgical repair attempt. The spinous processes of osteoporotic patients often are very strong (mostly cortical bone), so this approach works well even for osteoporotic patients. In some embodiments, methods of the present disclosure may be used for patients having severe or extreme vertebral compression. For example, patients having compressed vertebra which are ten percent (10%) of the normal height (e.g., a ninety percent (90%) compression fracture) can benefit from these methods. In some embodiments, a patient with some kyphosis can have more predictable results. The spinal implants help reduce the pressure on the compressed vertebra, but are not intended to alter or correct the kyphosis.

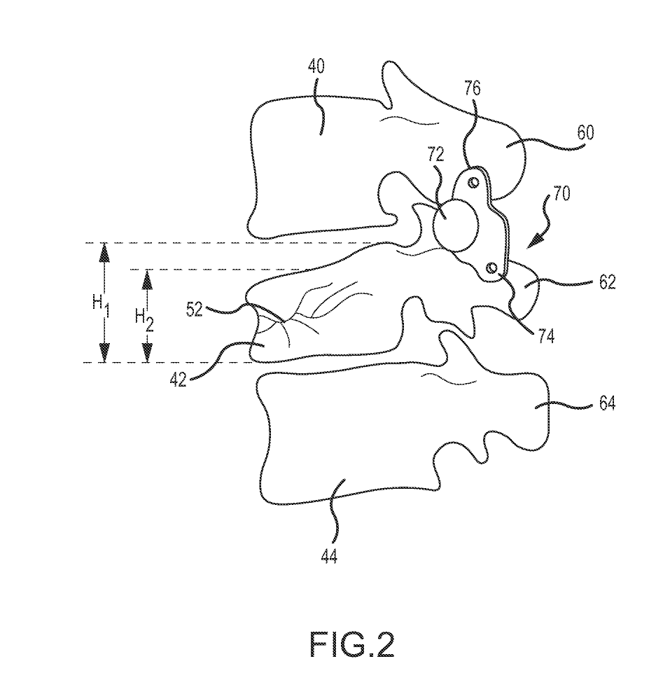

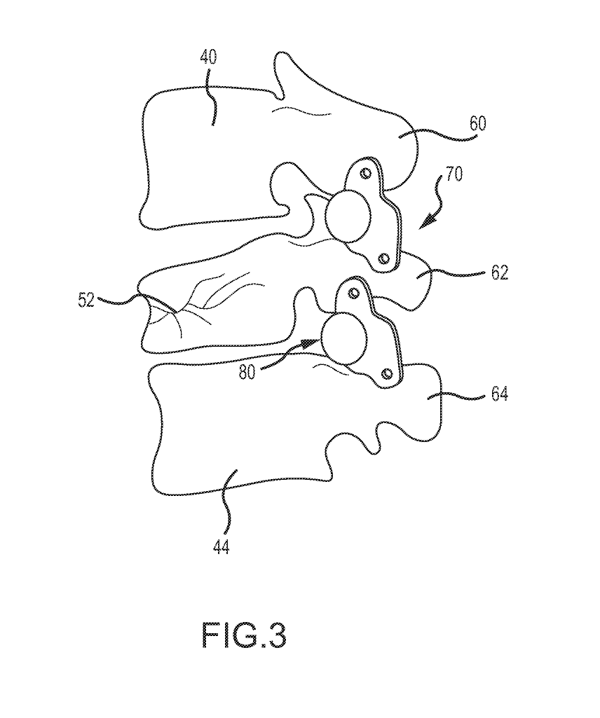

A wide range of implants may be used to implement the methods disclosed herein. As shown in FIG. 2, in one embodiment an implant 70 is coupled between vertebrae 40 and 42, and more specifically, is coupled between a spinous process 62 of vertebra 42 and a spinous process 60 of vertebra 40. In this example, vertebra 42 has a compressed height H2 which is less than a normal height H1. Normal height H1 is defined as the height of vertebra 42 if vertebra 42 did not have the compression fracture 52. In the depicted embodiment, implant 70 has a central member 72 disposed between spinous processes 60, 62. Central member 72 may be adapted to contain a bone growth promoting substance. For example, central member 72 may contain bone chips, bone morphogenic proteins, demineralized bone matrices, or a wide range of osteoinductive or osteogenic material. Implant 70 has a first extension 74 coupled to spinous process 62 and a second extension 76 coupled to spinous process 60. Extensions 74, 76 may be coupled to the spinous processes in any number of ways, including with screws or spikes inserted into the bone, or by coupling to mating extensions 74, 76 disposed on the opposing lateral sides of spinous processes 62, 60 (not shown).

Some methods of the present disclosure further provide a second implant 80, which is coupled between spinous process 62 and a spinous process 64 of vertebra 44. Implant 80 may have similar features as implant 70 including, for example, having a central portion with bone growth promoting substances and first and second extensions. Alternatively, implant 80 is coupled to spinous processes 62 and 64 using alternative mechanisms.

In some methods of the present disclosure, implants 70, 80 are coupled to previously untreated vertebra 42, and operate to maintain vertebra 42 in a stable position relative to vertebrae 40 and 44. This may be accomplished by inserting implants 70, 80 without distracting vertebrae 40, 42, 44, or without changing the superior-inferior spacing of spinous processes 60, 62, 64. In some embodiments, implant 70 is first coupled between the fracture vertebra 42 and the superior vertebra 40, after which implant 80 is coupled to the fractured vertebra 42 and the inferior vertebra 44. In other embodiments, implant 80 is coupled to the fractured vertebra 42 and the inferior vertebra 44 first, and thereafter implant 70 is coupled to the fractured vertebra 42 and the superior vertebra 40.

Spinous process implants for use according to some embodiments include a spacer and an extension extending outwardly from the spacer. The spinous process implant may be configured for insertion between adjacent spinous processes of the cervical, thoracic, and/or lumbar spine. The spacer may be provided in a variety of sizes to accommodate anatomical variation amongst patients and varying degrees of space correction. The spacer may include openings to facilitate tissue in-growth to anchor the spacer to the vertebral bodies such as tissue in-growth from the spinous processes. The spacer may be configured for tissue in-growth from superior and inferior spinous processes to cause fusion of the adjacent spinous processes. The openings may be relatively large and/or communicate to a hollow interior of the spacer. A hollow interior may be configured to receive bone growth promoting substances such as by packing the substances into the hollow interior. The openings may be relatively small and/or comprise pores or interconnecting pores over at least a portion of the spacer surface. The openings may be filled with bone growth promoting substances.

The spacer may have any suitable cross-sectional shape. For example, it may be cylindrical, D-shaped, C-shaped, H-shaped, include separated cantilevered beams, and/or any other suitable shape. The shape may include chamfers, fillets, flats, relief cuts, and/or other features to accommodate anatomical features such as for example the laminae and/or facets.

The extension may extend transversely from the spacer relative to a spacer longitudinal axis to maintain the spacer between adjacent spinous processes. A single extension may extend in one or more directions or multiple extensions may be provided that extend in multiple directions. One or more extensions may be adjustable longitudinally relative to one another and/or the spacer to allow the extensions to be positioned laterally relative to the spinous processes. A moveable extension may be provided that is movable axially relative to the spacer and another extension. Alternatively, a plurality of moveable extensions may be provided. For example, the extensions may clamp against the sides of the spinous processes to immobilize the spinous processes relative to one another and promote fusion between the adjacent vertebrae. The extensions may include fasteners engageable with the spinous processes. The fasteners may include sutures, wires, pins, straps, clamps, spikes, screws, teeth, adhesives, and/or other suitable fasteners. The fasteners may be integrated into the extensions or they may be modular. Modular fasteners may be adjustable, replaceable, and/or removable to allow tailoring of the kind and quality of fixation from rigid fixation to no fixation. The spacer, extensions, and/or fasteners may advantageously be made of different materials. For example, the spacer and extensions may be made of a relatively softer material while the fasteners may be made of a relatively harder material. For example, the spacer and/or extension may be made of a polymer and/or other relatively soft material and the fastener may be made of a metal and/or other relatively hard material.

Cerclage may be used to stabilize the spinous process implant and/or to provide other benefits. For example, wires, straps, bands, cables, cords, and/or other elongated members may encircle the pedicles, laminae, spinous processes, transverse processes, and/or other spinal structures. The cerclage may be relatively inextensible to provide a hard check to spine flexion or the cerclage may be relatively extensible to provide increasing resistance to flexion. The cerclage may be relatively flexible and drapeable such as a woven fabric or it may be relatively rigid such as a metal band. The cerclage may have shape memory properties that cause it to resume a prior set shape after implantation. The cerclage may be independent of the spinous process implant or may engage it. For example, the cerclage may pass through a hollow interior of the spinous process implant and/or engage the extension. The cerclage may be offset from the spacer and provide a tensioning force that uses the spacer as a fulcrum to offload the disc and/or open the disc space.

The implant may be supplemented with bone growth promoting substances to facilitate fusion of adjacent vertebrae between spinous processes, laminae, transverse processes, facets, and/or other spinal structures. The bone growth promoting substances may be spaced from the implant, placed adjacent the implant, sandwiched between the implant and underlying bone, placed inside the implant, coated onto the implant, and/or otherwise placed relative to the implant. If it is coated onto the implant it may cover the entire implant or only selected portions of the implant such as the extensions, fasteners, spinous process contacting portions of the spacer, and/or other portions.

As used herein, bone growth promoting substances may include bone paste, bone chips, bone strips, structural bone grafts, platelet derived growth factors, bone marrow aspirate, stem cells, bone growth proteins, bone growth peptides, bone attachment proteins, bone attachment peptides, hydroxylapatite, calcium phosphate, other suitable bone growth promoting substances, and/or combinations thereof.

The implant and any associated cerclage or other components may be made of any suitable biocompatible material including among others metals, resorbable ceramics, non-resorbable ceramics, resorbable polymers, and non-resorbable polymers. Some specific examples include stainless steel, titanium and its alloys including nickel-titanium alloys, tantalum, hydroxylapatite, calcium phosphate, bone, zirconia, alumina, carbon, bioglass, polyesters, polylactic acid, polyglycolic acid, polyolefins, polyamides, polyimides, polyacrylates, polyketones, fluoropolymers, and/or other suitable biocompatible materials and combinations thereof.

The spinous process implant may be used to treat spine disease in a variety of surgical techniques including superspinous ligament sacrificing posterior approaches, superspinous ligament preserving posterior approaches, lateral approaches, and/or other suitable approaches. The spinous process implant may be used to treat spine disease by fusing adjacent vertebrae or by preserving motion between adjacent vertebrae. It may include only an extension stop such as a spacer, only a flexion stop such as flexible cerclage elements, or both a flexion and extension stop, such as spinous process fasteners. The spinous process implant may be used to reduce loads on the facet joints, increase spinous process spacing, reduce loads on the disc, increase anterior disc spacing, and/or otherwise treat spine disease. Anterior effects may be accomplished by tensioning spine elements posterior to the spacer to apply a mechanical advantage to the spinal construct. Techniques for the spinal process implant may include leaving the tissues at the surgical site unmodified or modifying tissues such as trimming, rasping, roughening, and/or otherwise modifying tissues at the implant site.

The spinous process implant may have a dimension in a first direction that is less than a dimension in a second direction to aid in inserting the spinous process implant between adjacent spinous processes. For example, the spinous process implant may have a longitudinal axis and a leading end near one end of the longitudinal axis. The leading end may have a first dimension transverse to the longitudinal axis that is less than a second dimension transverse to the longitudinal axis such that the spinous process implant may be oriented with the first dimension aligned with the space between adjacent spinous processes to ease insertion and then oriented with the second dimension aligned with the spinous processes to space them apart a distance equal to the second dimension.

Insertion of spinous process implants may be facilitated by a set of instruments alternately engageable with one another to increase the interspinous space and engageable with a spinous process implant to help maneuver it between adjacent spinous processes.

Insertion of spinous process implants may be facilitated by an introducer insertable between adjacent spinous processes and able to engage a spinous process implant to help maneuver it between the adjacent spinous processes. The introducer may be rigid, flexible, or include both rigid and flexible portions. The introducer may engage the inside and/or the outside of the spinous process implant. The introducer may engage a relatively small portion or a relatively large portion of the spinous process implant. For example, the introducer may include a sleeve and/or trocar engageable with the inside or outside of the spinous process implant in nesting relationship. For example, a rigid sleeve may be positioned between adjacent spinous processes and then receive a spinous process implant such that when the sleeve is withdrawn the implant remains between the spinous processes. Such a sleeve may be initially inserted by installing a trocar in the sleeve. The introducer may include a flexible leader that is threadable between adjacent spinous processes to then draw the introducer and/or spinous process implant between the spinous processes. For example the introducer may include a sleeve with a relatively small diameter flexible leader extending from a first end and may be engageable with a spinous process implant at a second end such that it may be assembled with a spinous process implant and then the assembly drawn between the spinous processes by pulling on the leader. Alternatively, the introducer may be drawn between the spinous processes and then joined with the implant. The sleeve may be flexible to resiliently couple to the spinous process implant such as by compressing inside of the implant and/or stretching around the outside of the implant. The introducer may be solid or hollow. It may be rigid or flexible. It may be made of metal, plastic, and/or other suitable materials. The introducer may loosely engage the spinous process implant, as in a sliding relationship, or it may engage the spinous process implant such that the implant is constrained to move with the introducer. The introducer may engage the spinous process implant via a friction fit or a positive engagement.

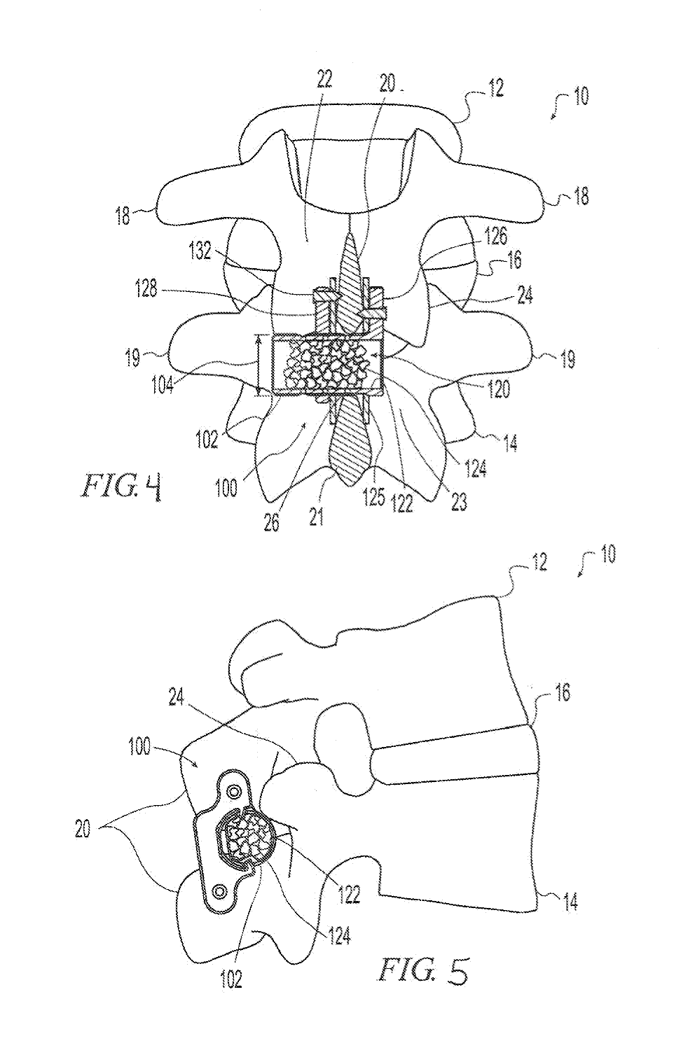

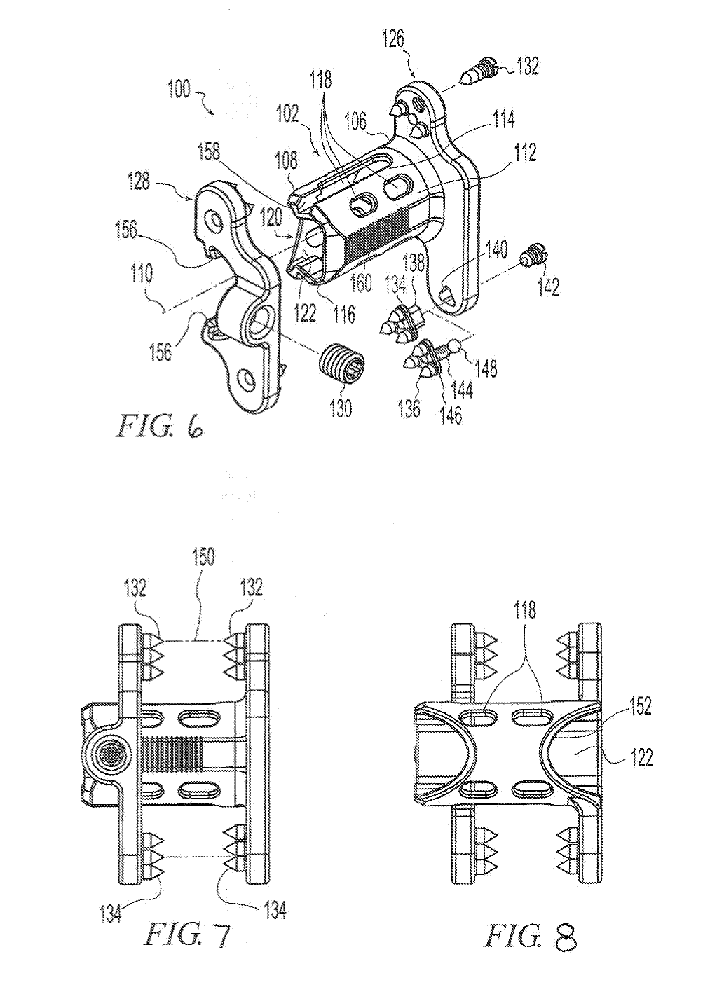

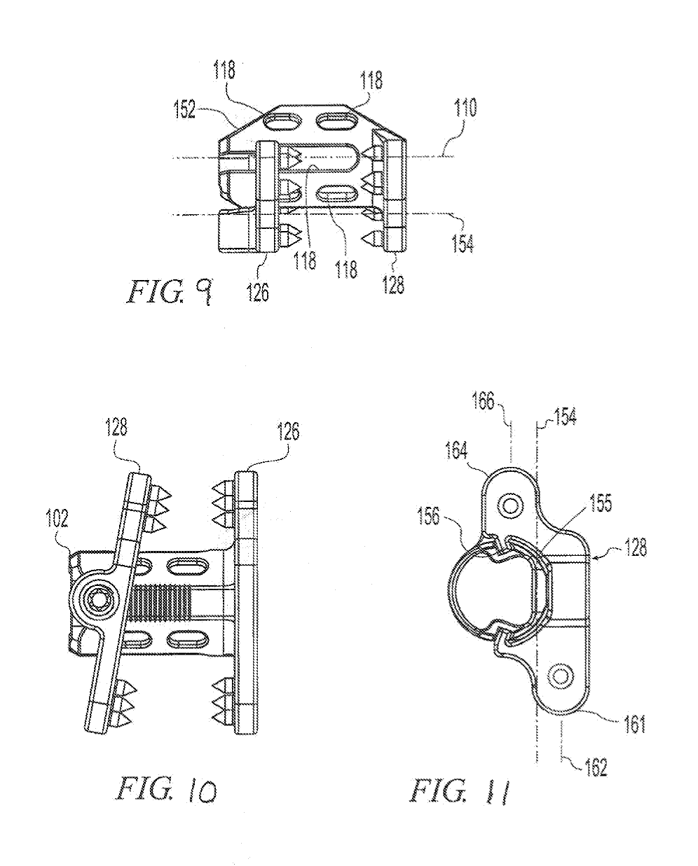

Further details on spinous process implants are provided with reference to FIGS. 4 and 5, which depict posterior and lateral views of a pair of adjacent vertebrae of the lumbar spine 10. A superior vertebra 12 is separated from an inferior vertebra 14 by a disc 16. Each vertebra includes a pair of transverse processes 18, 19, a posteriorly projecting spinous process 20, 21, and a pair of laminae 22, 23 connecting the transverse processes 18, 19 to the spinous process 20, 21. In addition to the connection through the disc 16, the vertebrae 12, 14 articulate at a pair of facet joints 24.

FIGS. 4-12 illustrate an exemplary spinous process implant 100 for use with methods of the present disclosure. The implant 100 includes a spacer 102 positioned between the spinous processes 20, 21. The geometry of the implant 100 is illustrated with the use of axes that define length (l), height (h), and width (w) directions for the spacer. When implant 100 is implanted in a patient, the height direction of the spacer 102 is generally oriented along the superior/inferior direction of the patient's anatomy, the width direction of the spacer 102 is generally oriented along the anterior/posterior direction of the patient's anatomy, and the length direction of the spacer 102 is generally oriented along the lateral/medial direction of the patient's anatomy.

The height 104 (FIG. 4) of spacer 102 limits how closely the spinous processes 20, 21 can move together. Thus, the spacer 102 maintains a minimum distance between the spinous processes 20, 21. In the case of spine disease involving posterior subsidence of the adjacent vertebra, insertion of the spacer 102 between the spinous processes 20, 21 will move the vertebrae apart and relieve pressure on nerve tissue and the facet joints 24.

As shown in FIG. 6, the spacer 102 includes a first end 106, a second end 108, and a longitudinal axis 110 extending from the first end to the second end. The spacer 102 has a sidewall 112, generally parallel to the longitudinal axis 110, including superior and inferior outer surfaces 114, 116. Transverse openings 118 (see also FIG. 9) communicate from the superior and inferior outer surfaces 114, 116 inwardly to facilitate tissue in-growth. The exemplary spacer 102 includes a hollow interior 120 bounded by an inner surface 122 such that the openings 118 communicate from the outer surfaces 114, 116 to the hollow interior 120. Bone growth promoting substances 124 are shown packed into the hollow interior 120 in FIGS. 4 and 5 to promote fusion of the vertebrae 12, 14 by bone growth between the spinous processes 20, 21.

The spinous process implant 100 further includes a first extension 126 projecting outwardly from the spacer 102 along the spacer height direction h and transversely to the longitudinal axis 110 to lie generally alongside the superior and inferior spinous processes 20, 21. Abutment of the first extension 126 with the spinous processes 20, 21 helps prevent lateral movement of spacer 102, thereby maintaining spacer 102 between the spinous processes 20, 21. In the exemplary spinous process implant 100, the first extension 126 is fixed relative to the spacer 102 and the implant includes a second extension 128 mountable to the spacer for axial movement relative to the first extension 126. The second extension 128 may be moved toward the first extension 126 to approximate the width of the spinous processes 20, 21 and better stabilize the implant 100. It is fixed in place by tightening a set screw 130 (FIG. 6) against the spacer 102. The extensions 126, 128 include fasteners 132, 134, 136 projecting from the extensions 126, 128 to engage the spinous processes 20, 21 to fix the spacer 102 to the spinous processes 20, 21. FIG. 4 depicts additional bone growth promoting substance in the form of a strips of bone 125 sandwiched between the extensions 126, 128 along the sides of the spinous processes 20, 21 to promote bone growth along the sides of the spinous processes to further enhance fusion of the vertebrae 12, 14. The extensions 126, 128 preferably extend inferiorly as well as superiorly from spacer 102 to optionally attach to the inferior spinous processes to immobilize the spinous processes 20, 21 relative to one another while fusion takes place.

Fasteners 132, 134, and 136 may take any suitable form. They may be made integral with the extensions 126, 128 such as by machining or casting them with the extensions or they may be formed separately and permanently attached to the extensions 126, 128. Fastener 132 is a sharpened spike that threadably engages the extension 126. The threaded engagement allows the fastener 132 to be replaced with a different fastener 132. For example, the fastener 132 may be replaced by one that has a different shape, a different size, a different material, or a different surface coating. The threaded engagement also allows the fastener 132 to be adjusted to extend by varying amounts from the extension 126 to vary how it engages the bone. Thus, the fastener 132 can be adjusted to fit differently shaped bones or to penetrate into a bone by varying amounts. For example, multiple threaded fasteners 132 can be adjusted to extend by different amounts to conform to curved or angled bone. Finally, the threaded engagement allows the user to remove the fastener 132 when fixation is not desired such as when it is desired to use implant 100 in a non-fusion procedure as an extension stop without limiting flexion.

As best seen in FIG. 6, fasteners 134 and 136 are provided as multi-spike pods allowing a plurality of spikes to be quickly adjusted, changed, or omitted. Fastener 134 includes a non-circular tab 138 engageable with a non-circular opening 140 in the extension 126. The non-circular engagement prevents the fastener 134 from rotating. The tab 138 may form a press-fit, snap-fit, or other suitable engagement with the opening 140. The tab 138 may be further secured by a supplemental screw 142. Fastener 136 includes a threaded shaft 144 threadably engaged with a base member 146 to allow the length of the fastener 136 to be adjusted. The shaft 144 engages the extension 126 in a rotating and pivoting manner such that the fastener 136 can be adjusted rotationally and angularly to engage the bone surface. In the illustrative embodiment, the shaft 144 terminates in a spherical ball 148 that engages the opening 140 in a ball-and-socket arrangement for three degrees of freedom. However, any mechanism that allows any number of degrees of freedom may be used. The fastener 136 may be allowed to move in use so that as the extension 126 is pressed toward a bone the fastener 136 adjusts to the angle of the bone surface. The fastener 136 may also be secured such as by screw 142 to adjust the tension in the joint and/or to lock the fastener 136 in a predetermined orientation.

FIG. 7 illustrates the axial relationship of fasteners on the opposing extensions 126, 128. In the illustrative implant 100, the fasteners 132 at the top of the implant 100 are shown aligned along a common axis 150 that is substantially perpendicular to extensions 126 and 128. The fasteners 134 at the bottom of the implant 100 are shown offset so that they can interleave if necessary as they are pressed into a bone. Any combination of fastener type, number, and alignment may be provided on the implant 100.

As seen in FIGS. 8 and 9, the ends 106, 108 of the spacer 102 include anterior chamfers 152. These chamfers 152 allow the ends 106, 108 to clear posteriorly facing structures of the vertebrae 12, 14 such as the facet joints 24. Also, as seen in FIGS. 8 and 9, the spacer 102 is offset anteriorly (in the spacer width direction w) relative to the extensions 126, 128 such that the longitudinal axis 110 of the spacer 102 is anterior of the midline 154 (FIGS. 9, 11) of the extensions 126, 128. The anterior offset of the spacer 102 allows it to fit deeply between the spinous processes 20, 21 while the extensions 126, 128 fit alongside the spinous processes 20, 21.

As best seen in FIGS. 6 and 11, the second extension 128 defines an aperture 155 conforming generally to the cross-sectional shape of the spacer 102. In the illustrative embodiment of FIGS. 4-12, the aperture 155 opens anteriorly to form a “C”-shape. Tabs 156 extend inwardly from the superior and inferior portions of the aperture to slidingly engage elongated slots 158 in the superior and inferior surfaces of the spacer 102. The second extension 128 can be translated longitudinally along the spacer length l toward and away from the first extension 126. Tightening the set screw 130 against the posterior side 160 of the spacer 102 forces the tabs 156 posteriorly against the sides of the slots 158 and locks the second extension 128 in place longitudinally. The posterior side 160 of the spacer 102 may be roughened as shown to better grip the set screw 130. The set screw 130 may also dig into the surface of the spacer 102 upon tightening to positively grip the spacer 102. The aperture 155 (FIGS. 6, 11) may conform closely to the spacer 102 to constrain the second extension 128 to generally parallel motion relative to the first extension 126. Alternatively, the aperture 155 may be larger than the spacer 102 by a predetermined amount to permit a predetermined amount of angular adjustment of the second extension 128 relative to the first extension 126 as shown in FIG. 10 to allow the extension 128 to adjust to the underlying bone surface.

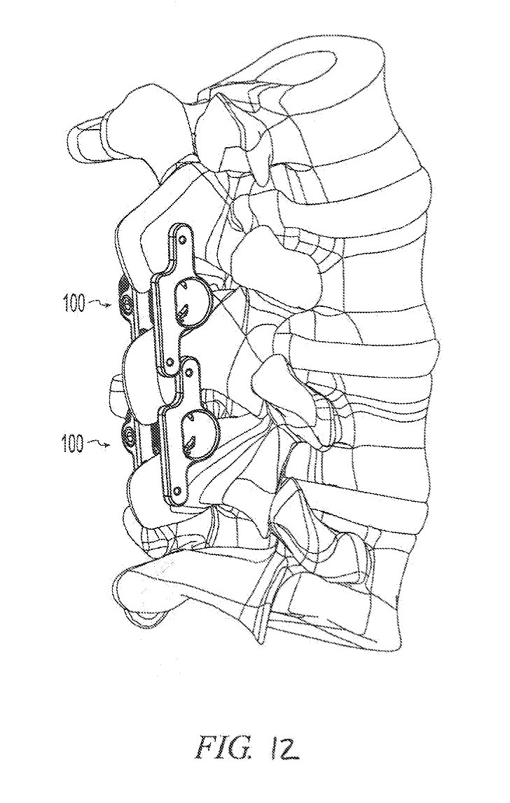

As best seen in FIG. 11, the second extension 128 includes a first inferior lobe 161 having a first lobe centerline 162 and a second superior lobe 164 having a second lobe centerline 166. In the illustrative embodiment, the first lobe centerline 162 and the second lobe centerline 166 are parallel and spaced apart so that the second extension 128 has a generally “Z”-shaped plan form. This shape allows the extension of one implant 100 to interleave, if necessary, with another implant 100 in a multilevel surgery (as shown in FIG. 12) to permit close spacing of the implants, and/or longer extension lobes for more extensive bone engagement. In addition, first inferior lobe 161 has a semi-circular convex shape that is generally complementary to a semi-circular superior concave surface 165 formed adjacent second superior lobe 164. Similarly, second superior lobe 164 has a semi-circular convex shape that is generally complementary in shape to a semi-circular inferior concave surface 163 formed adjacent first inferior lobe 161. As indicated in FIG. 11, first inferior lobe 161 is adjacent to inferior concave surface 163, and extension midline 154 is located between first inferior lobe 161 and inferior concave surface 163. Second superior lobe 164 is adjacent superior concave surface 165, and extension midline 154 is located between second superior lobe 164 and superior concave surface 165. Moreover, first inferior lobe radius r₁ is substantially equal to superior concave surface radius r₄, while second superior lobe radius r₃ is substantially equal to inferior concave surface radius r₂. As a result, when two implants are placed on adjacent spinal levels, the first inferior lobe 161 of the upper implant may be (but need not be, depending on what is medically indicated) interfitted into the superior concave surface 165 of the inferior implant. In addition, the second superior lobe 164 of the inferior implant may be interfitted into the inferior concave surface 163 of the superior implant. In the illustrative example of FIGS. 4-12, first lobe 161 and second lobe 164 form a unitary second extension 128. Although not separately depicted, first extension 126 also has complementary lobes that are similarly configured and oriented relative to one another.

As shown in FIG. 12, multiple spinous process implants 100 may be placed on adjacent levels of the spine. As illustrated in the figure, a first superior implant 100 is positioned with its spacer 102 between a first superior spinous process and a second intermediate spinous process, while a second inferior implant 100 is positioned with its spacer 102 between the second intermediate spinous process and a third inferior spinous process. The first extensions 126 of the superior and inferior implants are located on a first side of the patient's sagittal plane, while the second extensions 128 of the superior and inferior implants are located on a second side of the patient's sagittal plane.

In the illustrative embodiment of FIGS. 4-12, the extension lobe centerlines 162 and 166 are offset equidistantly from the midline 154 of the second extension 128. Although not separately shown, the first extension 126 is configured similarly. The centerlines 162 and 166 may vary from parallel and they may be offset asymmetrically to form different shapes to accommodate different vertebral anatomy. For example, the shape may be tailored for different portions of the spine 10. In the illustrative embodiment of FIGS. 4-12, the first extension 126 has the same shape as the second extension 128. However, the shape may be varied between the first and second extensions 126, 128.

Various methods, systems and devices for treating spinal fractures are disclosed. While detailed descriptions of one or more embodiments have been provided above, various alternatives, modifications, and equivalents are possible. Therefore, the above description should not be taken as limiting the scope of possible embodiments, which is defined by the appended claims.

What is claimed is:

1. A method of treating a spinal fracture, the method comprising:
identifying a vertebra having an untreated fracture, the vertebra having a spinous process;
coupling a first device to the spinous process, and further coupling the first device to a spinous process of a superior vertebra directly superior to the vertebra; and
coupling a second device to the spinous process, and further coupling the second device to an inferior vertebra directly inferior to the vertebra.

2. The method as in claim 1, wherein the untreated fracture comprises a compression fracture.

3. The method as in claim 2, wherein the compression fracture has reduced a height of the vertebra by at least about fifty percent (50%).

4. The method as in claim 2, wherein the compression fracture has reduced a height of the vertebra by at least about seventy-five percent (75%).

5. The method as in claim 1, further comprising applying a bone cement to the untreated fracture of the vertebra after coupling the first device to the spinous process.

6. The method as in claim 1, wherein the first device comprises a first member having first and second wings, the first wing for coupling to the spinous process and the second wing for coupling to the superior vertebra.

7. The method as in claim 6, wherein the first device comprises
a second member having third and fourth wings for coupling to the spinous process and the superior vertebra, respectively; and
an extension member adapted to extend between the spinous process and the spinous process of the superior vertebra.

8. The method as in claim 7, further comprising inserting a bone growth promoting substance in the extension member.

9. The method as in claim 1, wherein the first device comprises first and second members adapted to couple to opposing lateral sides of the spinous process, and to opposing lateral sides of the spinous process of the superior vertebra.

10. The method as in claim 1, further comprising placing a bone growth promoting substance between the spinous process and a spinous process of the superior vertebra.

11. The method as in claim 1 further comprising maintaining a distance between the spinous process and the spinous process of the superior vertebra prior to said coupling of the first device.

12. A method of treating a previously untreated spinal compression fracture, the method comprising:
identifying a vertebra having an untreated compression fracture, the vertebra having a spinous process;
selecting a first device adapted to couple to the spinous process and to a spinous process of a second vertebra adjacent the vertebra, wherein the first, device comprises:
a first component having a central portion and two extension portions extending from the central portion in generally opposite directions, the two extension portions being generally coplanar and having separate longitudinal axes;
a second component having a plurality of spikes;
an intermediate member adapted to be disposed between the spinous process and the spinous process of the second vertebra; and
a locking member adapted to couple the first and second components together with the intermediate member, wherein the first and second components are positioned on opposing lateral sides of the spinous process when coupled together; and
coupling the first device to the spinous process and to the spinous process of the second vertebra;
wherein the coupling of the first device operates to hold the vertebra in a set position relative to the second vertebra.

13. The method as claim 12, further comprising:
selecting a second device adapted to couple to the spinous process and to a spinous process of a third vertebra adjacent the vertebra, whereby the vertebra is positioned between the second and third vertebrae, the second device comprising:
a third component having a central portion and two extension portions extending from the central portion in generally opposite directions, the two extension portions being generally coplanar and having separate longitudinal axes;
a fourth component having a plurality of spikes;
a second intermediate member adapted to be disposed between the spinous process and the spinous process of the third vertebra; and
a second locking member adapted to couple the third and fourth components together with the second intermediate member, wherein the third and fourth components are positioned on opposing lateral sides of the spinous process when coupled together; and
coupling the second device to the spinous process and to the spinous process of the third vertebra.

14. The method as in claim 12, further comprising maintaining a distance between the spinous process and the spinous process of the second vertebra after the coupling of the first device to the spinous process and the spinous process of the second vertebra.

15. The method as in claim 12 further comprising coupling the first device to the spinous process and the spinous process of the second vertebra without distracting a gap between the spinous process and the spinous process of the second vertebra.

16. The method as in claim 12 further comprising coupling the second device to the spinous process and the spinous process of the third vertebra without distracting a gap between the spinous process and the spinous process of the third vertebra.
